(19) United States
(12) Patent Application Publication
(10) Pub. No.: US 2019/0247270 A1
(43) Pub. Date: Aug. 15, 2019

(54) INSTRUMENT FOR FACIAL PERCUSSIVE TREATMENT

(71) Applicant: Kimberly Carey, Naperville, IL (US)

(72) Inventor: Kimberly Carey, Naperville, IL (US)

(21) Appl. No.: 16/269,971

(22) Filed: Feb. 7, 2019

Related U.S. Application Data
(60) Provisional application No. 62/628,449, filed on Feb. 9, 2018.

(51) Int. Cl. A61H 23/02

(52) U.S. Cl. CPC A61H 23/02; A61M 16/0006

(57) ABSTRACT

A device for treating sinus congestion. The device includes a handle and a head. The handle has a longitudinal axis extending from a first end to a second end. The head is located at the first end of the handle. The head includes a longitudinal axis extending from a first end to a second end and being perpendicular to a longitudinal axis of the handle. The first end of the head includes a recess. The head is removably attached to the handle. The second end of the handle includes a removable cap that is metal.

### RELATED APPLICATIONS

This application claims priority from U.S. Provisional Patent Application Ser. No. 62/628,449 filed on Feb. 9, 2018, the entirety of which is incorporated herein by reference.

### FIELD OF THE INVENTION

This invention relates generally an instrument or device, and more particularly to a device for percussive treatment on patient's face to relieve sinus pressure/congestion.

### BACKGROUND OF THE INVENTION

There are currently a variety of treatment methods for relieving sinus congestion/pressure. For example, some treatment methods are based upon local medicinal agents or prescription drugs. Additionally, some treatment methods involve flushing of liquids, typically hot water, through the patient's sinuses. Still others involve devices inserted into the nasal cavity. Finally, surgery may be an option for treatment.

While these treatment methods are presumably effective for their intended purposes, they have drawbacks. For example, for many patients, it is often to avoid or minimize the use of or reliance on drugs. Similarly, some patients may not want to flush their sinuses with liquid or insert an implement into their sinuses. Finally, for some patients, surgery is too costly of an option. Other patients may belie that surgery to too drastic of a treatment method.

Therefore, there is a need for methods of treating sinus congestion/pressure that do not suffer from the drawbacks associated with conventional treatment processes.

### SUMMARY OF THE INVENTION

The present invention provides a solution to the foregoing problems and shortcomings of the treatment methods discussed above.

Specifically, the present invention provides a device for a percussive treatment on a patient's face to alleviate pain and provide relief from sinus pressure. Percussive treatment is currently used for treating some conditions. For example, for percussive treatment for an ailment of the lung, a treatment provider's hand is cupped as if to hold water but with the palm facing down and is clapped on the patient's chest or back. The cupped hand curves to the back or chest and provides a cushion of air to soften the clapping. After treatment, the patient expresses the loosened materials from the lungs by coughing.

The present invention recognizes that this treatment provides a potential low cost, minimally invasive treatment for sinus pressure/congestion. However, a cupped hand is often too large for treating a face of the patient. Therefore, the present invention provides a device that allows the percussive treatment to be applied to a patient's face to alleviate the sinus pressure/congestion.

Accordingly, the present invention, in at least one aspect, may be generally characterized as providing device for treating sinus congestion. The device has a handle with a longitudinal axis extending from a first end to a second end and a head at the first end of the handle. The head includes a longitudinal axis extending from a first end to a second end and is perpendicular to a longitudinal axis of the handle. The head and the handle may be discrete components that are removably secured. The first end of the head has a recess formed therein. The recess may have a hemispherical shape. The head may have a cylindrical shape. A bottom cap may be disposed at the second end of the handle. The bottom cap is a discrete component that may be removably secured to the handle. The second end of the head includes a convex hemispherical shape. The head is made from a rubber material.

The present invention may also be characterized, broadly, as providing device for treating sinus congestion which includes: a handle having a longitudinal axis extending from a first end to a second end; and, a head at the first end of the handle. The head includes a longitudinal axis extending from a first end to a second end and being perpendicular to a longitudinal axis of the handle. The first end of the head terminates with a recess. The head may be removably secured to the handle via an attachment member having a threaded shaft at a first end and a securement portion with a rough or non-smooth, textured surface at a second end. The recess may have a hemispherical shape. The head may have a cylindrical shape. The device may also include a bottom cap at the second end of the handle. The bottom cap may be a discrete component removably secured to the handle. The second end of the head may have a convex hemispherical shape. The head may be made from a rubber material.

The present invention may further be generally characterized, in at least one aspect, as providing device for treating sinus congestion which includes: a handle having a longitudinal axis extending from a first end to a second end, the handle being tapered at the first end and second end; a head removably secured at the first end of the handle, the head having a longitudinal axis extending from a first end to a second end and being perpendicular to a longitudinal axis of the handle, and the first end of the head terminating with a concave recess; an attachment member embedded in the head and having a threaded shaft for removably securing the head to the handle; and, a cap secured to the second end of the handle. The cap may be a different material than a material of the head. The cap may be metal. The head may be a rubber material. The second end of the head may include a convex hemispherical shape. The recess may have a hemispherical shape. The head may have a generally cylindrical shape.

In at least one aspect, the present invention may be characterized as providing a device for treating sinus congestion which includes: a handle having a longitudinal axis extending from a first end to a second end; a rubber head removably secured at the first end of the handle, the head having a longitudinal axis extending from a first end to a second end and being perpendicular to a longitudinal axis of the handle, the first end of the head terminating with a concave recess and the second end of the head forming a convex hemispherical shape; an attachment member embedded in the head and having a threaded shaft for removably securing the head to the handle; and, a metal cap secured to the second end of the handle. The handle may be tapered.

In yet another aspect of the present invention, the present invention provides a method of treating sinus pain by using a device according to any aspect or embodiment described herein.

Additional aspects, embodiments, and details of the invention, all of which may be combinable in any manner, are set forth in the following detailed description of the invention.

### DETAILED DESCRIPTION OF THE DRAWINGS

One or more exemplary embodiments of the present invention will be described below in conjunction with the following drawing figures, in which:

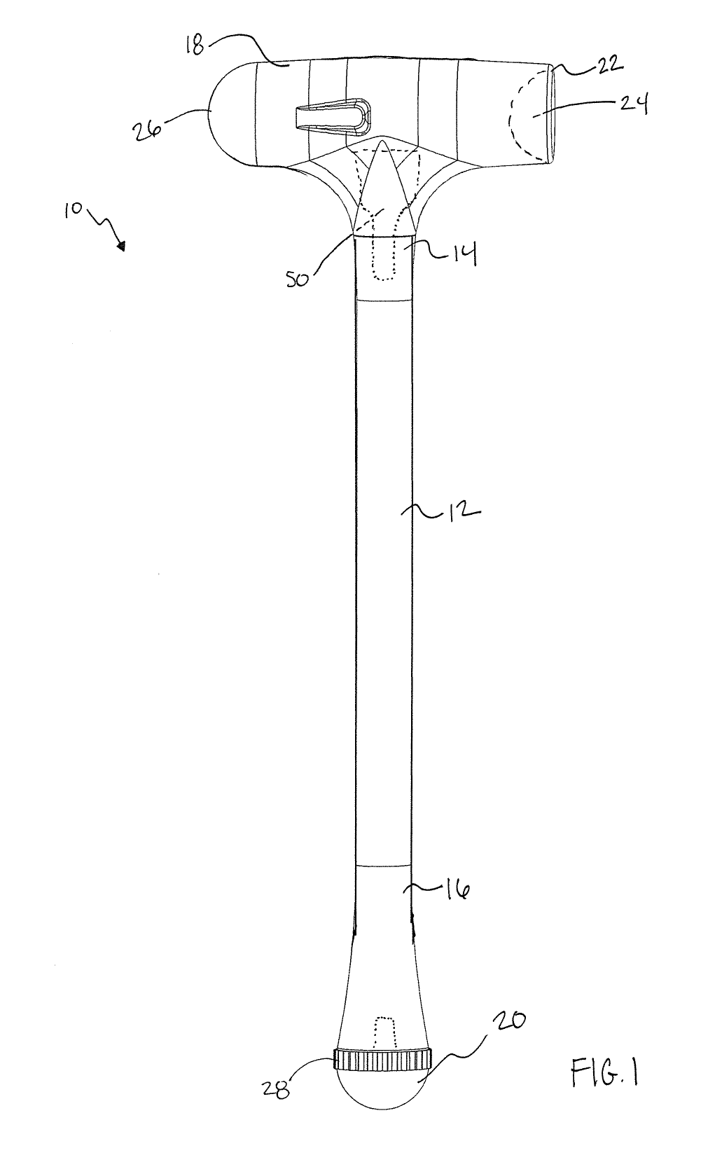

FIG. 1 shows a side view of a device according to the present invention;

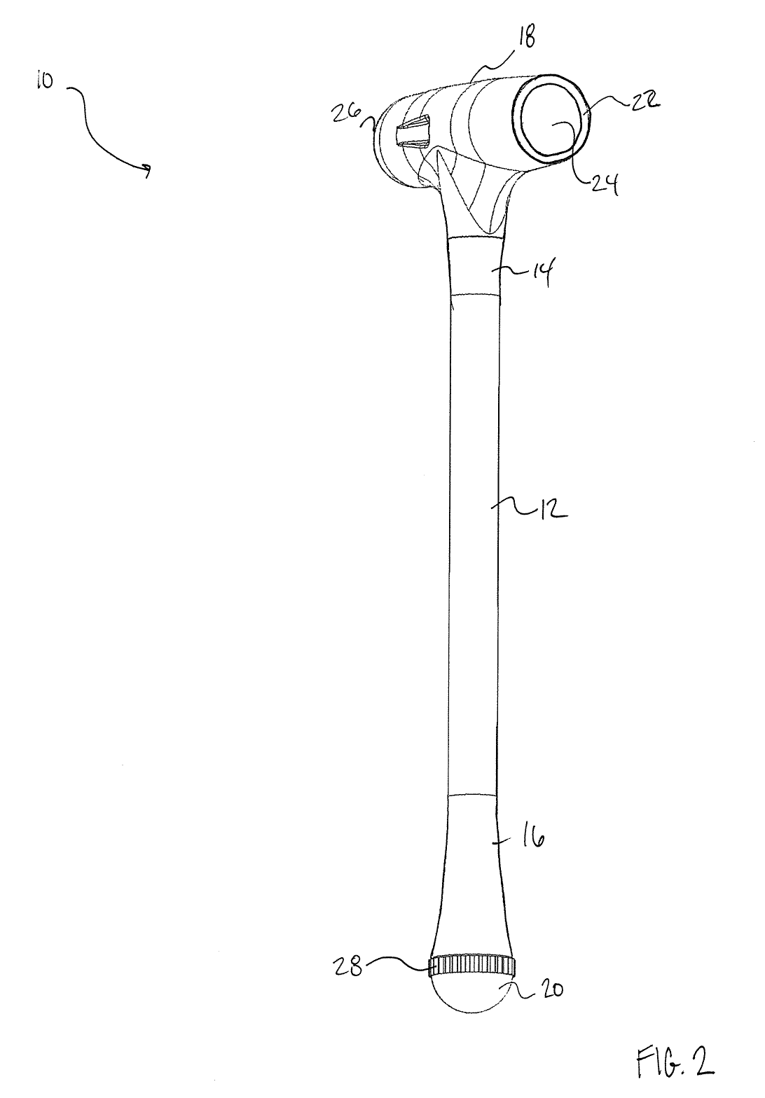

FIG. 2 shows a front and side view of the device shown in FIG. 1;

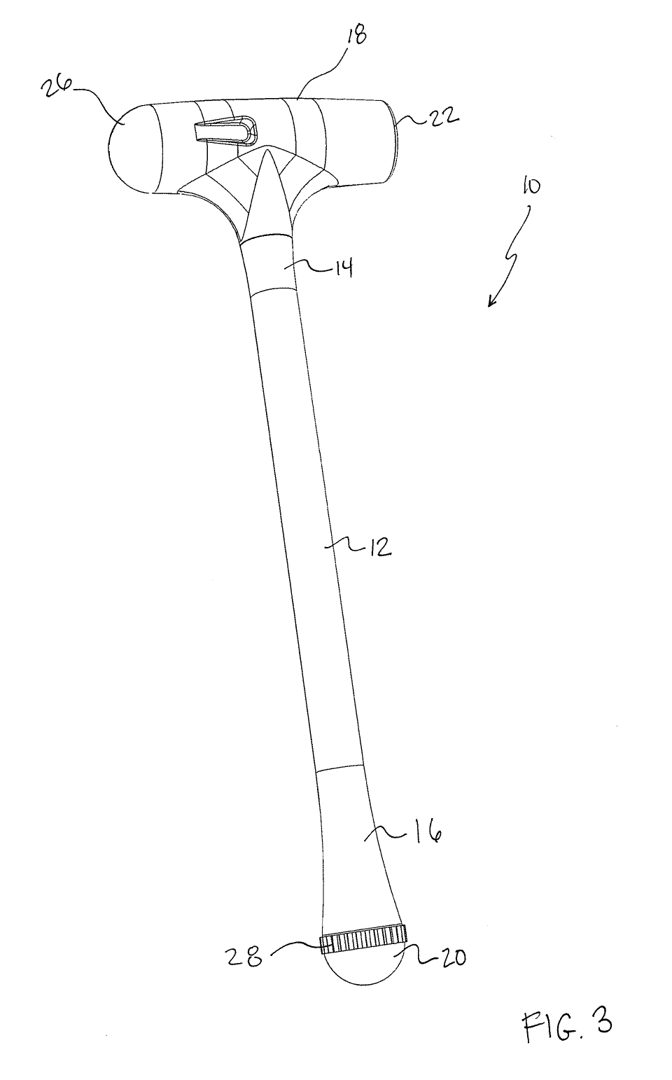

FIG. 3 shows a rear and side view of the device shown in FIG. 1;

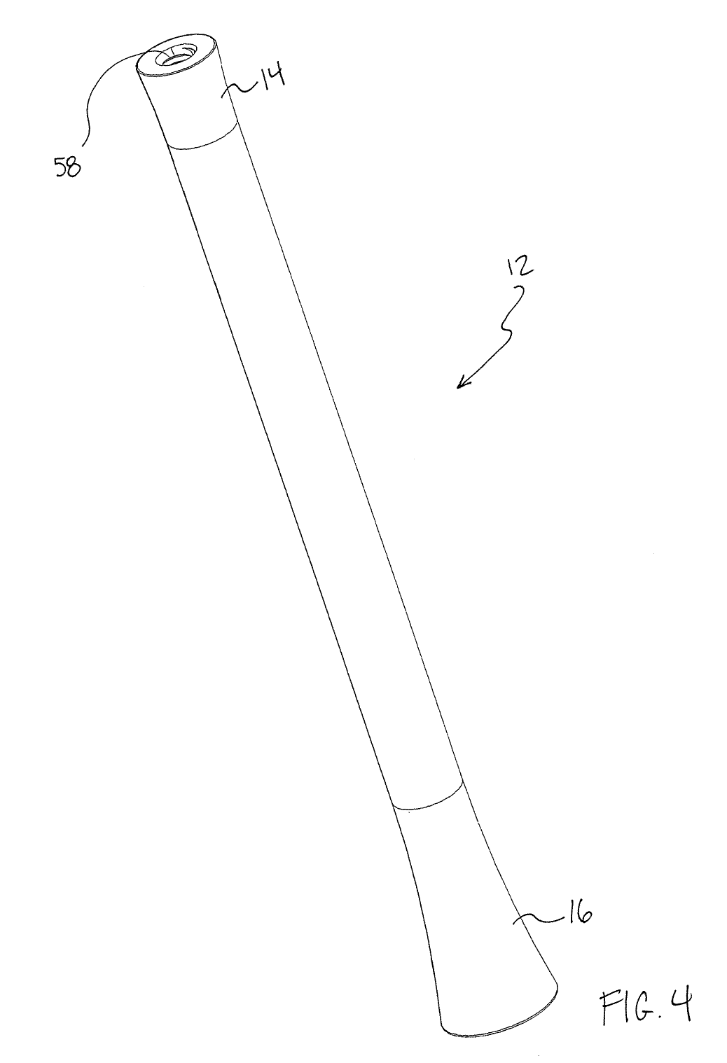

FIG. 4 shows a top and side view of the handle of the device shown in FIG. 1;

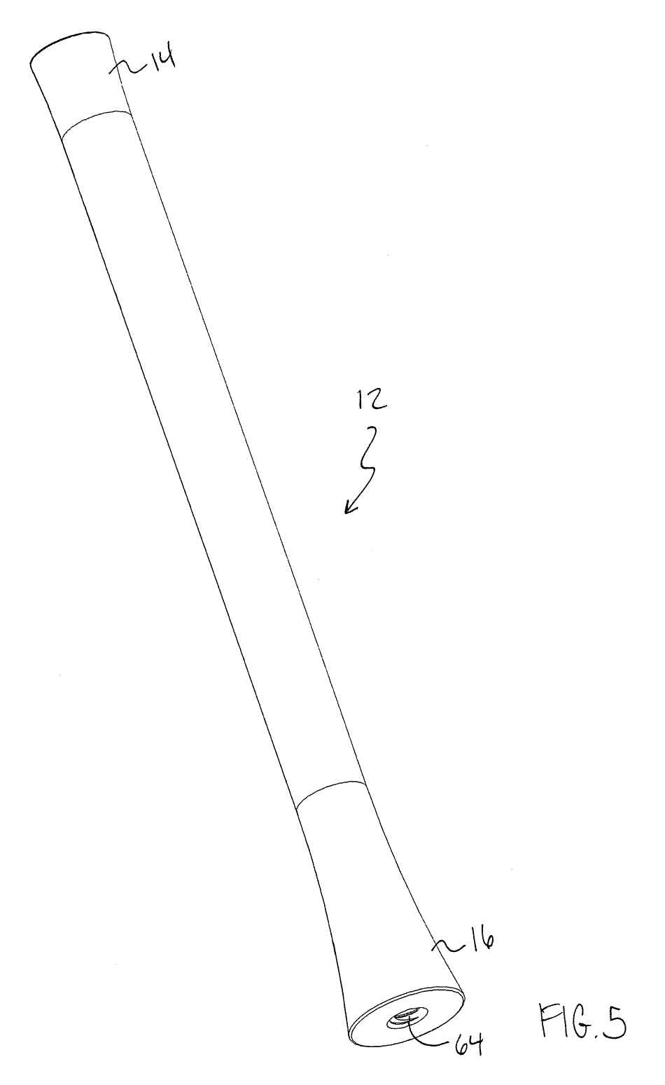

FIG. 5 shows a bottom and side view of the handle of the device shown in FIG. 1;

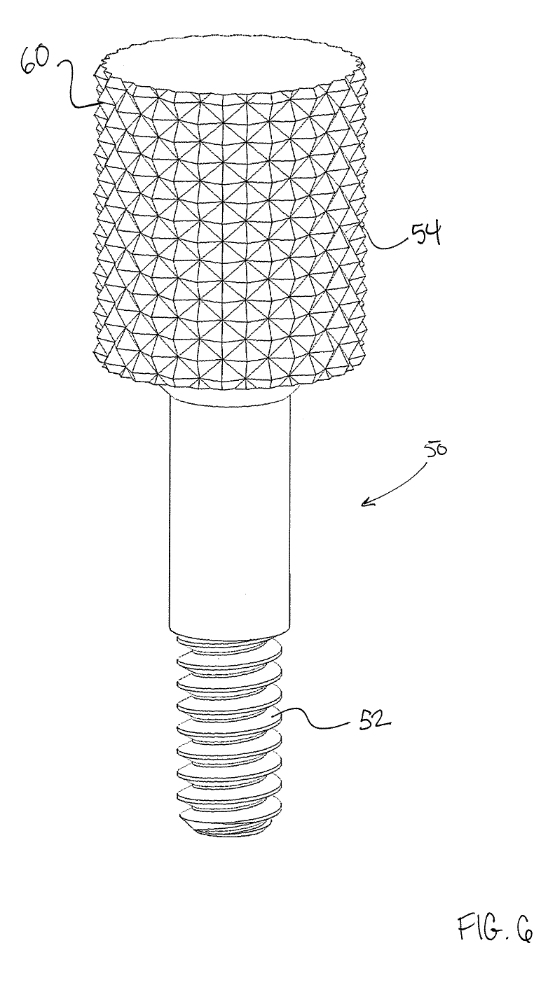

FIG. 6 shows side and top view of an attachment member for attaching the head to the handle of the device according to the present invention; and,

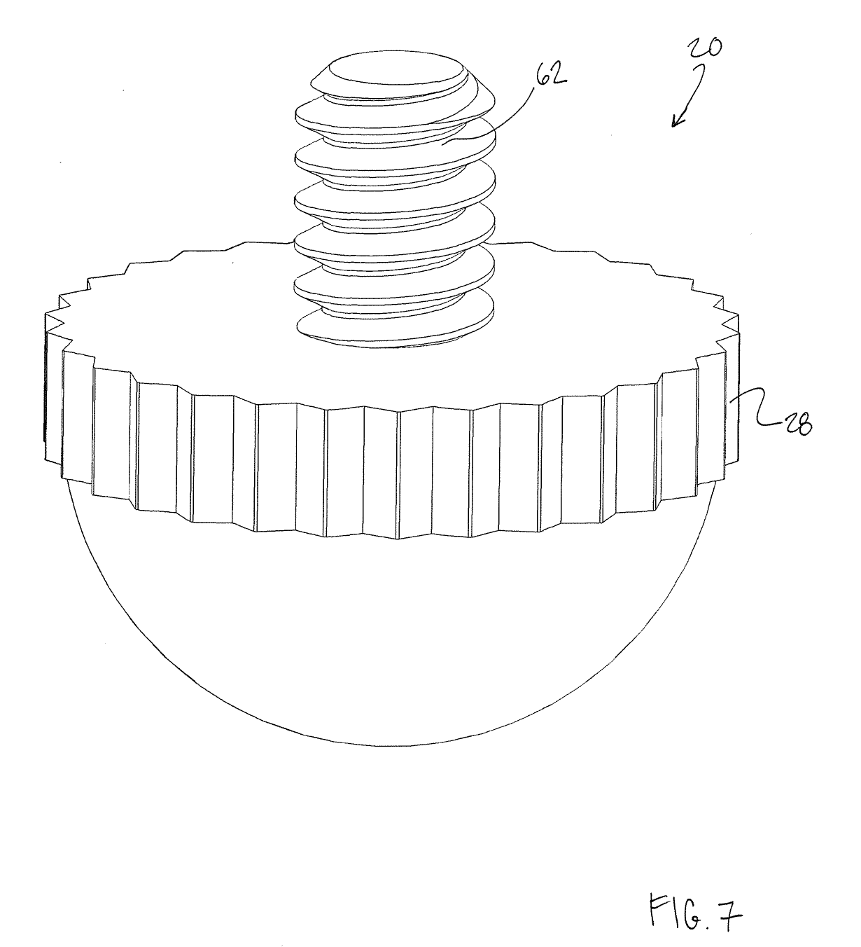

FIG. 7 shows a side and top view of the bottom cap of the device shown in FIG. 1.

### DETAILED DESCRIPTION OF THE INVENTION

With these general principles in mind, one or more embodiments of the present invention will be described with the understanding that the following description is not intended to be limiting.

As mentioned above, with reference to FIGS. 1 to 3, a device 10 according to the present invention includes an elongate handle 12 that extends between two ends 14, 16. The handle 12 is generally cylindrical having a length (extending along the longitudinal axis running between the two ends 14, 16) that is much greater than its diameter. The handle 12 is preferably made from a relatively rigid resin.

As shown in FIGS. 4 and 5, at the ends 14, 16 of the handle 12, the diameter of the handle 12 gradually increases to provide a taper to the handle 12 at the ends 14, 16. Returning to FIGS. 1 to 3, a head 18 is disposed at the first end 14 of the handle 12 and a bottom cap 20 is disposed at the second end 16 of the handle 12.

The head 18 is a discrete component securely attached to the handle 12. The handle 12 and the head 18 may be coupled together in any conventional manner suitable for the device 10 such as bonding, fasteners, glues, or other known fastening means.

In an exemplary embodiment, as shown in FIG. 1, the head 18 may be formed about or receive an attachment member 50. Turning to FIG. 6, a preferred attachment member 50 includes a bottom threaded shaft 52 and an upper securement portion 54. A cylindrical transition portion 56 may extend between the threaded shaft 52 and the securement portion 54. The threaded shaft 52 is received in a threaded bore 58 in the first end 14 of the handle 12. See, FIG. 4. The securement portion 54 is embedded within the head 18 and preferably has a rough, coarse, or non-smooth textured outer surface 60 to increase the purchase between the securement portion 54 (and thus the attachment member 50) and the head 18 surrounding the attachment member 50.

Returning to FIGS. 1 to 3, the head 18 of the device 10 also has a generally cylindrical shape (having a length greater than a width). Preferably, the length of the head 18 (measured along the longitudinal axis of the head 18) is less than the length of the handle 12. However, the diameter of the head 18 is greater than the diameter of the handle 12 (apart from any taper at the first end 14 of the handle 12). This size/mass ratio between the head 18 and the handle 12 provides the momentum for the device 10 when being used for a percussive treatment.

Preferably, the head 18 is made from a material that is relatively rigid and holds it shape, but not so rigid that it causes discomfort to a patient. For example, certain rubbers may be utilized. Preferred rubbers include rubbers with a low durometer value, for example less than 70A shore, or less than 60A shore.

A first end 22 of the head 18 (along the longitudinal axis of the head 18) includes an inwardly formed recess 24 at the end 22. Thus, the head 18 has a recess 24 at one of its ends 22 (i.e., the head terminates in a recess 24). As depicted in FIGS. 1 and 2, the recess 24 is formed by a concave cavity extending away from the first end 22 of the head 18 towards a second end 26 of the head 18. The recess 24 at the first end 22 preferably has a hemispherical shape. Other shapes may be used for the recess 24 provided that the shape provides a percussive effect when the head 18, and more particularity the outer edge of the recess 24, is brought in contact with the face of the patient.

The second end 26 of the head 18 has or forms a convex hemispherical shape. The second end 26 is configured to massage the face of the patient after first end 22 has been utilized to provide a percussive treatment. Accordingly, it should be appreciated that the depicted shape of the second end 26 is merely exemplary and other shapes and configurations of the second end 26 are contemplated. Preferably, the shape of the second end 26 of the head 18 includes a smooth surface, but in some instances a non-smooth textured surface may be present.

The bottom cap 20 at the second end 16 of the handle 12 preferably is a separate or discrete component from the handle 12. Turning to FIG. 7 the bottom cap 20 includes a threaded shaft 62 to secure the bottom cap 20 and to a threaded bore 64 in the handle 12. See, FIG. 5 Other connecting structures may be used. It is also contemplated that the bottom cap 20 is integrally formed with the handle 12. However, when the bottom cap 20 and the handle 12 are separate components, the bottom cap 20 is configured to removed and put in a low temperature environment (such as a freezer or refrigerator) to lower a temperature of the bottom cap 20. Accordingly, it is also preferred that the bottom cap 20 is formed from a metal or another similar material that has a good specific heat capacity so its temperature is easily lowered and maintained at a reduced temperature (relative to room temperature) for a suitable period of time. The material of the outer surface of the bottom cap 20 should be non-corrosive and inert to oils, ointments, and other medicaments that might be applied during treatment. To facilitate removal and attachment of the bottom cap 20, the bottom cap 20 includes a knurled surface 28.

Accordingly, for a percussive treatment to be performed on a patient's face, the device 10 is used by either a clinician (or other individual) or the patient directly. The device 10 is grasped by the handle 12 and the first end 22 of the head 18 is lightly clapped (i.e. tapped), repeatedly, on the face, near the location of the patient's sinuses (i.e., around the eyes and nose). In order to use the advantages of the mass/size ratio between the head 18 and the handle 12, the device 10 is preferably grasped closer to the second end 16 of the handle than the first end 14 of the handle 12. As discussed above, the recess 24 softens the impact of the head 18 and provides a percussive effect to loosen material in the sinus.

After a few minutes of clapping with the first end 22 of the head 18, the second end 26 of the head 18 may be used to massage the same area to help move any loosened materials out of sinus so that it is able to be expressed by the patient. Similarly, the bottom cap 20 (if previously cooled) may be used to massage the same area and the cooled bottom cap 20 provides an anti-swelling or numbing effect. Additionally, the bottom cap 20 is utilized to apply ointments, oils, or other medicaments to the patient's skin.

With the present device 10, a patient is able to obtain a treatment for sinus congestion/treatment without requiring any drugs or other ingested medicines. Additionally, the device provides a treatment that does not require flushing the sinus with liquid. Furthermore, the treatment methods with the device 10 are significantly less invasive than those treatment methods that insert a device into the sinus of the patient, including surgery. Moreover, the device 10 allows a patient to self-treat, providing a significant advantage over conventional treatment processes that require another person to treat the patient.

While at least one exemplary embodiment has been presented in the foregoing detailed description of the invention, it should be appreciated that a vast number of variations exist. It should also be appreciated that the exemplary embodiment or exemplary embodiments are only examples, and are not intended to limit the scope, applicability, or configuration of the invention in any way. Rather, the foregoing detailed description will provide those skilled in the art with a convenient road map for implementing an exemplary embodiment of the invention, it being understood that various changes may be made in the function and arrangement of elements described in an exemplary embodiment without departing from the scope of the invention as set forth in the appended claims and their legal equivalents.

What is claimed is:

1. A device for treating sinus congestion, the device comprising:
a handle having a longitudinal axis extending from a first end to a second end; and,
a head at the first end of the handle, the head having a longitudinal axis extending from a first end to a second end and being perpendicular to a longitudinal axis of the handle, the first end of the head terminates with a recess.

2. The device of claim 1 wherein the head is removably secured to the handle via an attachment member having a threaded shaft at a first end and a securement portion with a rough surface at a second end.

3. The device of claim 1 wherein the recess has a hemispherical shape.

4. The device of claim 1 wherein the head has a cylindrical shape.

5. The device of claim 1 further comprising:
a bottom cap at the second end of the handle.

6. The device of claim 5 wherein the bottom cap is a discrete component removably secured to the handle.

7. The device of claim 1 wherein the second end of the head comprises a convex hemispherical shape.

8. The device of claim 1 wherein the head is made from a rubber material.

9. A device for treating sinus congestion, the device comprising:
a handle having a longitudinal axis extending from a first end to a second end, the handle being tapered at the first end and second end;
a head removably secured at the first end of the handle, the head having a longitudinal axis extending from a first end to a second end and being perpendicular to a longitudinal axis of the handle, the first end of the head terminating with a concave recess;
an attachment member embedded in the head and having a threaded shaft for removably securing the head to the handle; and,
a cap secured to the second end of the handle, the cap being a different material than a material of the head.

10. The device of claim 9, wherein the cap is metal.

11. The device of claim 10, wherein the head comprise a rubber material.

12. The device of claim 9, wherein the second end of the head comprises a convex hemispherical shape.

13. The device of claim 9 wherein the recess has a hemispherical shape.

14. The device of claim 13 wherein the head has a cylindrical shape.

15. A device for treating sinus congestion, the device comprising:
a handle having a longitudinal axis extending from a first end to a second end;
a rubber head removably secured at the first end of the handle, the head having a longitudinal axis extending from a first end to a second end and being perpendicular to a longitudinal axis of the handle, the first end of the head terminating with a concave recess and the second end of the head forming a convex hemispherical shape;
an attachment member embedded in the head and having a threaded shaft for removably securing the head to the handle; and,
a metal cap secured to the second end of the handle.

16. The device of claim 15 wherein the handle is tapered.
